(19) United States
(12) Patent Application Publication
(10) Pub. No.: US 2021/0298943 A1
(43) Pub. Date: Sep. 30, 2021

(54) CARE TRAVEL SYSTEM AND METHOD

(71) Applicant: Ashraf Elkossei, Oak Lawn, IL (US)

(72) Inventor: Ashraf Elkossei, Oak Lawn, IL (US)

(21) Appl. No.: 16/835,774

(22) Filed: Mar. 31, 2020

(51) Int. Cl. A61F 7/08; A61F 7/10; A47G 9/10; A47C 16/00

(52) U.S. Cl. CPC A61F 7/08; A61F 7/10; A47G 9/1045; A61F 2007/0088; A47G 9/1081; A47G 9/1036; A47C 16/00

(57) ABSTRACT

A care travel system which provides comfort and relief from headaches. It holds an ice pack or other chilled items against the head, neck, and shoulders. The device counteracts fevers when facing an illness. It effectively eliminates the need to take medication, which can make people sleepy and groggy or cause an allergic reaction. As designed it prevents soreness and discomfort that typically manifests during travel.

### BACKGROUND OF THE INVENTION

The following includes information that may be useful in understanding the present disclosure. It is not an admission that any of the information provided herein is prior art nor material to the presently described or claimed inventions, nor that any publication or document that is specifically or implicitly referenced is prior art.

### TECHNICAL FIELD

The present invention relates generally to the field of beds of existing art and more specifically relates to pillows adapted to accommodate disparate article(s).

### RELATED ART

People often experience headaches, illnesses, and other discomfort during travel. As a result, they may take one of the various headache medicines that are currently on the market, but these medicines may leave people feeling groggy or tired, while other people may be allergic to these formulas. Additionally, people may not have options during transit, whether on a plane, on a bus, or in a car. A suitable solution is desired.

U.S. Pat. No. 4,783,866 to Ethel D. Simmons et al. relates to a therapy pillow with removable therapeutic gel pack. The described therapy pillow with removable therapeutic gel pack includes a cold pack specially constructed and contoured to provide intimate contact with the occipital region of the head. The pillow constructed and contoured to fit partially around the neck and tapered so that the surface portion of the pillow will apply an even force to the occipital region of the head. A pocket provided in the pillow receives a specially contoured container filled with a temperature retaining material providing a cold pack which will relieve migraine or muscular contraction headaches when applied to the head. The specially constructed pocket is lined with a moisture resistant material and is easily and quickly opened and closed for receiving the cold pack.

### SUMMARY OF THE INVENTION

In view of the foregoing disadvantages inherent in the known beds and pillows art, the present disclosure provides a novel care travel system. The general purpose of the present disclosure, which will be described subsequently in greater detail, is to provide an efficient and effective care travel system.

A care travel system is disclosed herein, the system in a preferred embodiment comprising: an arcuate cushioned pillow having a body with an interior curved portion, an exterior curved portion, a top, and a bottom; an insulated pouch designed to receive at least one item located around the interior curved portion; a cover with at least one fastener. The at least one fastener allows for access to the insulated pouch such that a user can insert and alternately remove the at least one item at will. The preferred embodiment has the at least one fastener comprising a zipping fastener; wherein the zipping fastener comprises a plurality of teeth; however other suitably equivalent fastening means may be used. The at least one item is insertable to promote comfort for the user (or alternately for use in storing the item). The insulated pouch restricts relative movement of heat for instances wherein the at least one item is a heating pack, or a cooling pack. The arcuate cushioned pillow comprises a crescent-shaped neck pillow; wherein the cover with the at least one fastener envelops the body during use; the cover comprising fabric and the body comprising cushioning material. The cushioning material is deformable. Relationally speaking the interior curved portion of the body is adjacent a neck of the user during use. A kit is also disclosed herein.

A method of use for the care travel system is also disclosed herein, the method comprising the steps of: providing a care travel system, (the system comprising: an arcuate cushioned pillow having a body with an interior curved portion, an exterior curved portion, a top, and a bottom; an insulated pouch designed to receive at least one item located around the interior curved portion; a cover with at least one fastener); opening the at least one fastener to access the insulated pouch; and inserting the at least one item for comforting use. The method may further comprise the step of removing the at least one item for comforting use and replacing as desired.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and methods of use for the present disclosure, a care travel system, constructed and operative according to the teachings of the present disclosure.

FIG. 1 is a perspective view of the care travel system during an ‘in-use’ condition, according to an embodiment of the disclosure.

FIG. 2 is a perspective view of the care travel system of FIG. 1, according to an embodiment of the present disclosure.

FIG. 3 is a perspective view of the care travel system of FIG. 1, according to an embodiment of the present disclosure.

FIG. 4 is a perspective view of the care travel system of FIG. 1, according to an embodiment of the present disclosure.

FIG. 5 is a flow diagram illustrating a method of use for the care travel system, according to an embodiment of the present disclosure.

The various embodiments of the present invention will hereinafter be described in conjunction with the appended drawings, wherein like designations denote like elements.

### DETAILED DESCRIPTION

As discussed above, embodiments of the present disclosure relate to a pillow or bed and more particularly to a care travel system as used to improve the comfort of traveling.

Generally, Care Travel soothes headaches, fevers, or other minor medical inconveniences during travel. This innovative product may comprise a crescent-shaped neck pillow, made from a soft, comfortable fabric. Along the interior curve of the pillow can be an insulated zippered pouch, designed to hold an ice pack or other items, like a chilled water bottle or canned beverage. Further, the neck pillow can be offered in assorted sizes, as well as various prints, colors, and designs, to accommodate a wide range of users. The exact specifications may vary upon manufacturing.

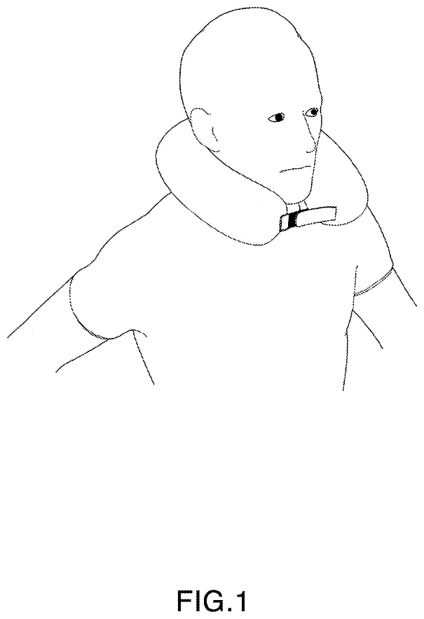

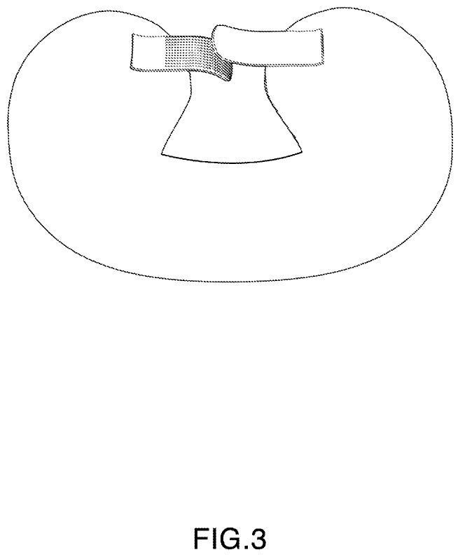

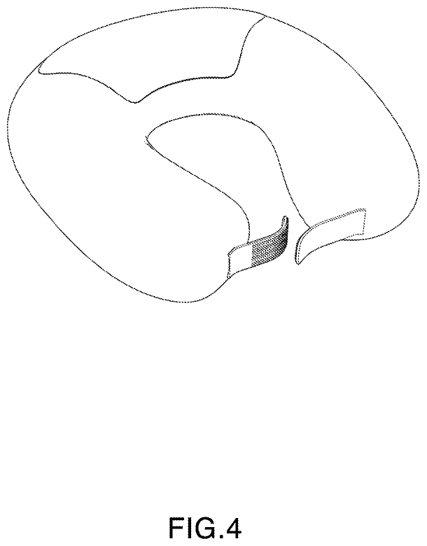

Referring now more specifically to the drawings by numerals of reference, there is shown in FIGS. 1-4, various views of a care travel system 100.

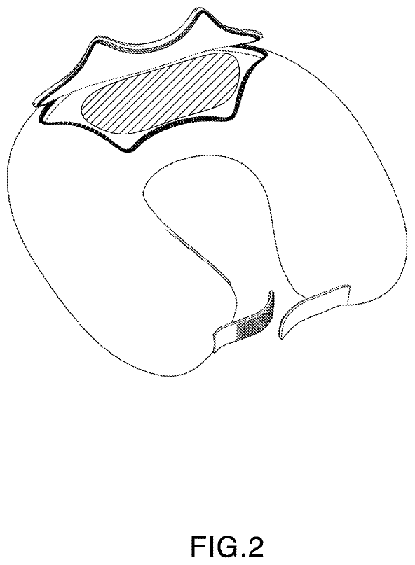

FIG. 1 shows a care travel system 100 during an ‘in-use’ condition 50, according to an embodiment of the present disclosure. Here, the care travel system 100 may be beneficial for use by a user to provide comfort during travel episodes. Referring now to FIGS. 2-4, as illustrated, the care travel system 100 may include an arcuate cushioned pillow 110 having a body 112 with an interior curved portion 114, an exterior curved portion 116, a top 118, and a bottom 120; an insulated pouch 115 designed to receive at least one item 130 located around the interior curved portion 114; and a cover 122 (preferably soft stretchable fabric material) with at least one fastener 126. The at least one fastener 126 allows for access to the insulated pouch 115 such that a user can insert and alternately remove the at least one item 130 at will.

The at least one fastener 126 preferably comprises a zipping fastener 128; wherein the zipping fastener 128 comprises a plurality of teeth. Alternate fastening means may comprise other suitably equivalent means such as snaps, clips, buttons, mating sleeves or the like.

The at least one item 130 is insertable to promote comfort for the user; wherein the at least one item 130 may be a heating pack or a cooling pack or other such comforting device. The heating pack emits radiant heat and is insulated by the insulated pouch 115 to promote extended use of the heating pack. When used the cooling pack emits cold and is insulated by the insulated pouch 115 to promote extended use of the cooling pack. Those with ordinary skill in the art will now appreciate that upon reading this specification and by their understanding the art of insulating as described herein, methods of insulation will be understood by those knowledgeable in such art. The at least one item 130 may alternately be a travel-item such as a canned or bottled beverage or any suitably sized item desired to be stored and transported via the care travel system 100.

The arcuate cushioned pillow 110 comprises a crescent-shaped neck pillow; wherein the cover 122 with the at least one fastener 126 envelops the body 112 during use. Cover 122 may be removable and washable; wherein the cover 122 preferably comprises fabric. The body 112 comprises cushioning material; wherein the cushioning material is deformable. Relationally speaking the interior curved portion 114 of the body 112 is adjacent a neck of the user during use as shown in FIG. 1.

According to one embodiment, the care travel system 100 may be arranged as a kit 105. In particular, the care travel system 100 may further include a set of instructions. The instructions may detail functional relationships in relation to the structure of the care travel system 100 such that the care travel system 100 can be used, maintained, or the like, in a preferred manner.

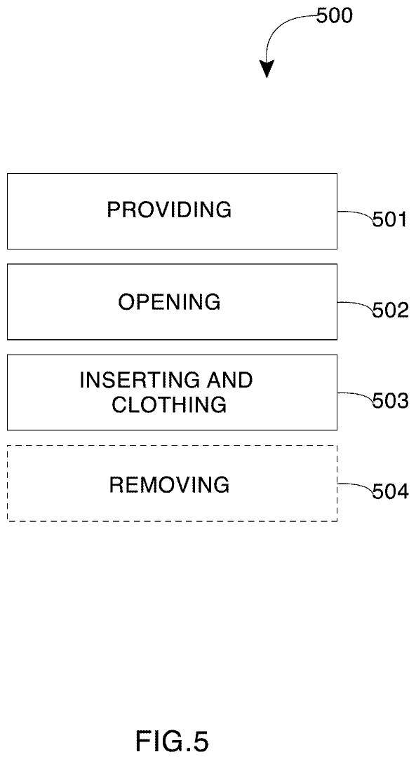

FIG. 5 is a flow diagram illustrating a method of use of the care travel system 100, according to an embodiment of the present disclosure. In particular, the method for use 500 may include one or more components or features of the care travel system 100 as described above. As illustrated, the method for use 500 may include the steps of: step one 501, providing a care travel system, (the system comprising: an arcuate cushioned pillow having a body with an interior curved portion, an exterior curved portion, a top, and a bottom; an insulated pouch designed to receive at least one item located around the interior curved portion; a cover with at least one fastener); step two 502, opening the at least one fastener to access the insulated pouch; and; step three 503, inserting the at least one item for comforting use and closing. The method may further comprise the step 504 of removing the at least one item for comforting use and replacing as desired.

It should be noted that step 504 is an optional step and may not be implemented in all cases. Optional steps of method of use 500 are illustrated using dotted lines in FIG. 5 so as to distinguish them from the other steps of method of use 500. It should also be noted that the steps described in the method of use can be carried out in many different orders according to user preference. The use of “step of” should not be interpreted as “step for”, in the claims herein and is not intended to invoke the provisions of 35 U.S.C. § 112(f). It should also be noted that, under appropriate circumstances, considering such issues as design preference, user preferences, marketing preferences, cost, structural requirements, available materials, technological advances, etc., other methods for use of the travel care system 100 are taught herein.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A care travel system comprising:
a cushioned pillow having
a body with
an interior curved portion, and
an exterior curved portion,
an insulated pouch located around the interior curved portion and designed to receive at least one item;
and
a cover with at least one fastener disposed on or around the insulated pouch.

2. The care travel system of claim 1, wherein the at least one fastener comprises a zipping fastener.

3. The care travel system of claim 1, wherein the at least one item is insertable to promote comfort for a user.

4. The care travel system of claim 1, wherein the at least one item is a heating pack.

5. The care travel system of claim 4, wherein the heating pack emits radiant heat and is insulated by the insulated pouch to promote extended use of the heating pack.

6. The care travel system of claim 1, wherein the at least one item is a cooling pack.

7. The care travel system of claim 6 wherein the at least one item is a cold can or bottle of a beverage.

8. The care travel system of claim 6, wherein the cooling pack absorbs heat and is insulated by the insulated pouch to promote extended use of the cooling pack.

9. The care travel system of claim 1, wherein the at least one item is a travel-item.

10. The care travel system of claim 1, wherein the cushioned pillow comprises a crescent-shaped neck pillow.

11. The care travel system of claim 1, wherein the cover with the at least one fastener envelops the body during use.

12. The care travel system of claim 11, wherein the cover comprises fabric.

13. The care travel system of claim 1, wherein the body comprises cushioning material.

14. The care travel system of claim 13, wherein the cushioning material is deformable.

15. The care travel system of claim 1, wherein the interior curved portion of the body is adjacent a neck of a user during use.

16. The care travel system of claim 1, wherein the insulated pouch restricts relative movement of heat.

17. A care travel system, the system comprising:
an arcuate cushioned pillow having
a body with
an interior curved portion,
an exterior curved portion,
a top, and
a bottom;
an insulated pouch designed to receive at least one item located around the interior curved portion;
a fabric cover with at least one fastener; and
wherein the at least one fastener allows for access to the insulated pouch such that a user can insert and alternately remove the at least one item;
wherein the at least one fastener comprises a zipping fastener;
wherein the zipping fastener comprises a plurality of teeth;
wherein the at least one item is a heating pack or a cooling pack;
wherein the pillow comprises a crescent-shaped neck pillow;
wherein the cover envelops the body during use;
wherein the body comprises cushioning material;
wherein the cushioning material is deformable; and
wherein the interior curved portion of the body is adjacent a neck of the user during use.

18. The care travel system of claim 17, further comprising set of instructions; and
wherein the care travel system is arranged as a kit.

19. A method comprising the steps of:
providing the care travel system of claim 1
opening the at least one fastener to access the insulated pouch; and
inserting the at least one item.

20. The method of claim 19, further comprising the step of removing the at least one item.
